(19) United States
(12) Patent Application Publication
(10) Pub. No.: US 2018/0271589 A1
(43) Pub. Date: Sep. 27, 2018

(54) MICRONEEDLE TREATMENT APPARATUS AND VARIABLE FREQUENCY RF TREATMENT SYSTEM

(71) Applicant: Shenzhen Peninsula Medical Co., Ltd., Shenzhen (CN)

(72) Inventors: Xiaobing LEI, Shenzhen (CN); Yongsheng LIANG, Shenzhen (CN)

(73) Assignee: Shenzhen Peninsula Medical Co., Ltd., Shenzhen (CN)

(21) Appl. No.: 15/850,596

(22) Filed: Dec. 21, 2017

(30) Foreign Application Priority Data
Mar. 24, 2017 (CN) 201710185945.9
Jul. 19, 2017 (CN) 201710591421.X

(51) Int. Cl. A61B 18/14; A61M 37/00

(52) U.S. Cl. CPC A61B 18/1477; A61B 2018/00071; A61M 37/0015

(57) ABSTRACT

A microneedle treatment apparatus includes a microneedle treatment head, which further includes a housing having a receiving chamber and a treatment port communicating with the receiving chamber; a microneedle device disposed in the receiving chamber and including a microneedle base and at least one microneedle fixed to the microneedle base and extending toward the treatment port; and a cold end conducting portion fixed in the receiving chamber and including a contact end exposed to the treatment port and a heat-conducting end which conductively connects to the contact end and a cold output end of a cooling output device. The microneedle treatment apparatus can perform cold treatment through the contact between the cold end conducting portion and the treated area, so that the side effect of microneedle therapy can be reduced. A variable frequency RF treatment system includes an inverter circuit, an impedance matching circuit and a controller.

### CROSS-REFERENCE TO RELATED APPLICATIONS

The present application claims priority to Chinese Patent Application Nos. CN 201710591421.X filed on Jul. 19, 2017 and CN 201710185945.9 filed on Mar. 24, 2017. The disclosures of these applications are hereby incorporated by reference in their entirety.

### BACKGROUND

Current microneedle array radiofrequency (RF) treatment may lead to epidermal injury or scald, and in the course of the treatment, timely cold treatment cannot be conducted. Moreover, the downtime after completion of the treatment is relatively long and high skin temperature affects the treatment effect.

RF treatment technology is widely applied in the field of medical treatment. Because different frequencies have different electrical conductivity in biological tissues, heat penetration and thermal dispersion area vary from frequency to frequency. Especially in the field of skin cosmetology, target treatment tissue may have a dermis layer, a fat layer, a superficial fascia layer, a muscle layer and so on. The electrical conductivity of these tissues varies more than 10 times.

### SUMMARY

The inventors of the present disclosure have recognized that, in the microneedle array RF treatment of dermis, it may be needed to control the tissue heating area so that the clinical side effect can be reduced. During the treatment of fat, the tissue heating area needs to be expanded to achieve the maximum treatment efficacy. When the face is treated with single-needle radiofrequency, the wrinkle treatment for superficial fat requires as little thermal dispersion as possible to prevent the skin from being injured in the treatment process, while the treatment for the a deeper-layer fat needs as large thermal dispersion area as possible to improve treatment efficacy.

Therefore, how to improve the operation frequency of a RF treatment system according to different target tissues being treated so that they can have different heat penetration and thermal dispersion areas which can make the treatment more precise and efficient remains to be resolved by the personnel in this field.

One object of the present disclosure is to provide a microneedle treatment head which can perform cold treatment to the treated area.

In one aspect of the present disclosure, a microneedle treatment head is provided for a microneedle treatment apparatus with a cooling output portion. The microneedle treatment head includes: a housing having a receiving chamber and a treatment port communicating with the receiving chamber; a microneedle device disposed in the receiving chamber and including a microneedle base and at least one microneedle, wherein the microneedle(s) is/are fixed to the microneedle base and extend toward the treatment port; and a cold end conducting portion fixed in the receiving chamber and including a contact end exposed to the treatment port and a heat-conducting end which connects to the contact end with thermal conductive connection, wherein the heat-conducting end is also connected to a cold output end of a cooling output device and the connection is thermal conductive.

In some embodiments of the present disclosure, the contact end has pinholes for threading the microneedles.

In some embodiments, the contact end has an outward-facing contact area which is flush with the front end of the treatment port.

In some embodiments, the microneedle base is adjustably installed inside the receiving chamber, so that the microneedles can extend to and retract into the treatment port.

In some embodiments, the treatment port is provided with a negative pressure chamber which is disposed with an open end toward the outside of the treatment port for adsorbing the area that needs to be treated; the contact end is disposed in the center of the treatment port and the negative pressure chamber extends along the interior periphery of the treatment port; and the negative pressure chamber is provided with a negative pressure through-hole to connect with a negative pressure output portion.

In another aspect of the present disclosure, a microneedle treatment apparatus is provided. The microneedle treatment apparatus include a microneedle treatment head and a treatment device body, the microneedle treatment head is fastened to the treatment device body; wherein the microneedle treatment head includes a housing having a receiving chamber and a treatment port communicating with the receiving chamber; a microneedle device disposed in the receiving chamber and including a microneedle base and at least one microneedle, wherein the microneedles are fixed to the microneedle base and extend toward the treatment port; and a cold end conducting portion fixed in the receiving chamber and including a contact end exposed to the treatment port and a heat-conducting end which connects to the contact end with thermal conductive connection, wherein the heat-conducting end also connects to the cold output end of the cooling output device with thermal conductive connection. The treatment device body includes the cooling output device, which is connected to the heat-conducting end with thermal conductive connection.

In some embodiments in the present disclosure, the cooling output portion includes a semiconductor cooling assembly and a radiator, wherein a cold end of the semiconductor cooling assembly connects to the heat-conducting end and the connection is thermal conductive, and a hot end of the semiconductor cooling assembly connects to the radiator and the connection is thermal conductive.

In some embodiments, the radiator includes a heat-conducting pipe and a heat sink. The heat-conducting pipe is connected to the hot end of the semiconductor cooling assembly for conducting the heat of the hot end of the semiconductor cooling assembly to the heat sink, wherein the heat-conducting pipe is a liquid-vapor heat pipe.

In some embodiments, the treatment device body has a detachable connection with the microneedle treatment head. The treatment device body includes a drive for sliding the microneedle base.

In some embodiments, the treatment device body includes the negative pressure output portion. A negative pressure through-hole connects the negative pressure chamber to the negative pressure output portion.

The present disclosure provides a cold end conducting portion on the microneedle treatment head. In the course of treatment, the cold end conducting portion has a thermally conductive connection with the cooling output portion, so that after contacting the treated area, the cold end conducting portion can perform cold treatment to the area, and thus side effects of microneedle therapy can be reduced.

In another aspect of the present disclosure, a variable frequency RF treatment system is provided to solve the problem of inefficiency and low precision of treatment caused by existing RF treatment systems, which fail to adjust output frequency according to different treatment tissues.

In order to achieve the above-mentioned aim and other related ones, the present disclosure provides a variable frequency RF treatment system, which includes an inverter circuit with various frequency outputs, an impedance matching circuit and a controller. After a power signal is input in the inverter circuit, the inverter circuit and the impedance matching circuit are connected and they are connected to the controller. The inverter circuit switches its frequency according to a frequency conversion signal transmitted by the controller. According to the received actual impedance, the impedance matching circuit matches an optimal impedance and conducts the radiofrequency output.

In some embodiments of the present disclosure, the inverter circuit includes a plurality of switches, each correspondingly connecting to a fixed frequency.

In some embodiments, the impedance matching circuit performs impedance matching by switching to different impedance matching circuits. The impedance matching circuit includes a plurality of switches, each connecting to a corresponding impedance.

In some embodiments, the impedance matching circuit performs impedance matching by current modulation.

In some embodiments, the impedance matching circuit performs impedance matching by combining impedance matching circuits and current modulation.

In some embodiments, the variable frequency RF treatment system includes an acquisition circuit, which acquires the actual impedance of user's skin tissue and sends it to the controller.

In some embodiments, the power signal is generated by a power controller which connects to the controller to receive its setting.

In some embodiments, the frequencies are set respectively according to different designations of application circuits.

In some embodiments, the frequency is set according to the input of a user interface.

In some embodiments, the frequency is set according to the detection mode of impedance matching.

In another aspect, a treatment system is provided including variable frequency RF treatment circuits described above, and the microneedle treatment apparatus. Variable frequency RF electromagnetic field can be delivered to the patent through the microneedles to achieve treatment effects.

As aforementioned, the various embodiments of the variable frequency RF treatment system can not only carry out RF treatment on different skins and detect the impedance value of the skin tissue, but also switch to the optimal output frequency according to the different skin conditions, thus improving the treatment effect and the treatment precision.

### BRIEF DESCRIPTION OF DRAWINGS

In order to clearly illustrate the embodiments in the present disclosure, the accompanying drawings that need to be used in the description of the embodiments will be introduced briefly. Apparently, the following accompanying drawings are just some embodiments of the present disclosure, for those skilled in the art, they can acquire other accompanying drawings based on structures shown in these accompanying drawings on the premise of not paying creative labor.

FIG. 1 shows a perspective view of a microneedle treatment head provided in some embodiments of the present disclosure;

FIG. 2 illustrates a sectional view of a microneedle treatment head provided in some embodiments of the present disclosure;

FIG. 3 illustrates an exploded view of a microneedle treatment head provided in some embodiments of the present disclosure;

FIG. 4 illustrates a sectional view of a microneedle treatment apparatus provided in some embodiments of in the present disclosure;

FIG. 5 shows a structural diagram of a variable frequency RF treatment system provided in some embodiments of the present disclosure; and

FIG. 6 shows a structural diagram of a frequency output mode provided in some embodiments of the present disclosure.

In the drawings:

1, controller; 2, inverter circuit; 3, impedance matching circuit; 4, sampling circuit; 5, power controller; 6, input end; 7, DC input; 8, radiofrequency output; 10, housing; 11, receiving chamber; 12, treatment port; 13, installation port; 20, cold end conducting circuit; 21, contact end; 22, heat-conducting end; 30, microneedle device; 31, microneedle base; 32, microneedles; 40, negative pressure chamber; 41, negative pressure through-hole; 50, thermal conductive silicone; 60, support device; 61, fixed base; 62, sliding housing; 63, back stand; 100, microneedle treatment head; 200, treatment device body; 211, contact area; 212, pinhole; 220, pushrod apparatus; 230, semiconductor cooling assembly; 231, cold end; 232, hot end; 240, radiator; 241, heat-conducting pipe; 242, heat sink; 2421, heat sink bottom; 2422, heat sink top; 251, negative pressure connecting pipe; 252, filter; 300, microneedle treatment apparatus; A, input interface; B, application component; C, impedance detection.

Combined with embodiments, the achievement of objects, the functional characteristics and the advantages of the present disclosure will be further described with reference to the accompanying drawings.

### DETAILED DESCRIPTION

Various embodiments of the present disclosure are described below with specific examples, and other advantages and effects of the present disclosure can be easily understood by those skilled in the field of technology from the contents disclosed in this specification. Apparently, the described embodiments are only a part of embodiments in the present disclosure, rather than all of them. The present disclosure can also be implemented or applied through different specific embodiments, and various details of the specification can also be modified or changed based on different viewpoints and applications without departing from the spirit of the present disclosure. Based on the embodiments in the present disclosure, all the other embodiments acquired by those skilled in the art on the premise of not paying creative labor are in the protection scope of the present disclosure. It should be noted that, on the premise that there is no conflict, the following embodiments and the features in the embodiments can be combined together.

It's necessary to note that if the embodiments in the present disclosure have directional instructions (such as up, down, left, right, front, back . . . ), then these directional instructions are only used for explaining relative position and movement among each element under a certain posture (as shown in the accompanying drawings), if this certain posture is changed, then the directional instruction will be changed correspondingly.

In addition, if there are descriptions such as “first” and “second” in the embodiments of the present disclosure, then these descriptions are only used for describing object rather than instructing or implying their relative importance or the number of technical features. Thus, features defined as “first” or “second” include explicitly or implicitly at least one of these features. Moreover, technical proposal of each embodiment can be combined, based on knowledge of those of ordinary skill in the art, when there is mutual contradiction or impossibility of the combination of technical proposals, the combination of these technical proposals should be considered as non-existent, and also out of the scope of the present disclosure.

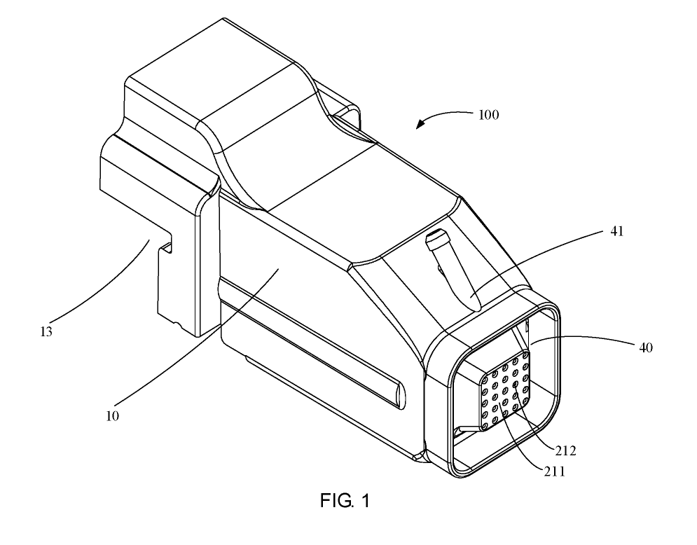

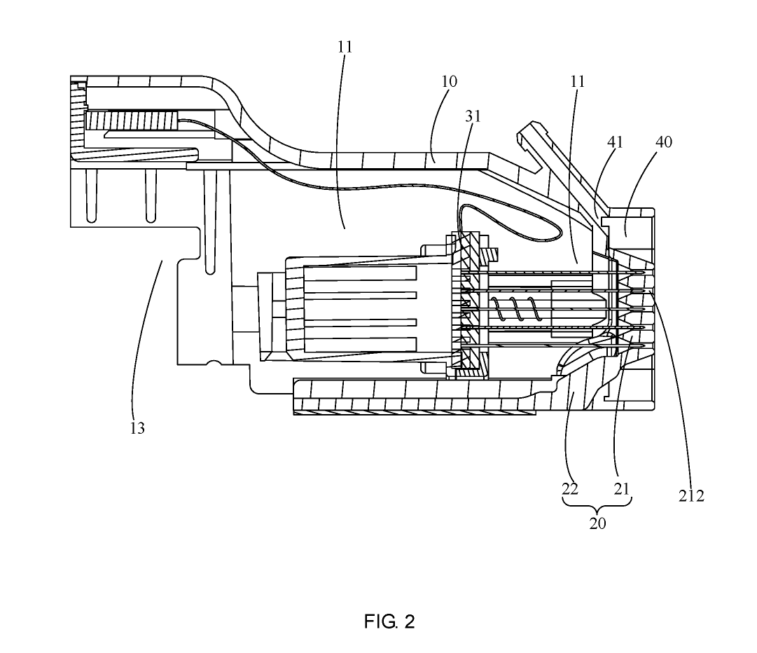

As shown in FIGS. 1-2, a microneedle treatment head 100 for a microneedle treatment apparatus with a cooling output circuit, includes a housing 10 having a receiving chamber 11 and a treatment port 12 communicating with the receiving chamber 11; a microneedle device 30 disposed in the receiving chamber 11 and including a microneedle base 31 and at least one microneedle 32, wherein the microneedles 32 are fixed to the microneedle base 31 and extend toward the treatment port 12; and a cold end conducting portion 20 fixed in the receiving chamber 11 and including a contact end 21 exposed to the treatment port 12 and a heat-conducting end 22 which connect to the contact end 21 with thermal conductive connection, wherein the heat-conducting end 22 is connected to a cold output end of a cooling output device with thermal conductive connection.

The treatment port 12 is disposed in the right side of the microneedle treatment head 100, the contact end 21 and the heat-conducting end 22 are both plate shaped. The inside of the receiving chamber 11 is for placing the elements inside the microneedle treatment head 100. The area that needs to be treated fits to the treatment port 12 of the microneedle treatment head 100, thus the area that needs to be treated can abut against the contact end 21 of the cold end conducting portion 20. The cold end conducting portion 20 is connected with a cooling output portion, thus the cold end conducting portion 20 can conduct the heat in the area being treated to the cooling output portion. As a result, cold treatment can be done to the area being treated. The temperature of the area being treated can be controlled through controlling the cooling output portion. Thus the treated area can be kept in an appropriate low temperature state, and microneedle treatment can achieve a better effect, and the side effect of pain and escharosis etc., caused by microneedle treatment can be reduced.

The treatment of the microneedles 32 is through piercing into the area that needs to be treated and outputting radiofrequency under the skin. Generally, in the course of microneedle treatment, the required emission time of radiofrequency is usually above 100 ms, between 500 ms-10000 ms in general. For example, the pulse emission time for osmidrosis treatment is usually above 2000 ms, wherein the microneedles 32 can be configured on the microneedle base 31 according to the need, and the microneedles 32 can immediately extend from the treatment port 12 directly or from the receiving chamber 11.

The cold end conducting portion 20 is made of metal, which can be made of red copper, aluminum, stainless steel, etc. In view of weight, aluminum or aluminum alloy are more preferable. It can be understood that when the cold end conducting portion is medal, insulation should be disposed between the microneedles 32 and the cold end conducting portion 20, such as disposing an insulation sleeves in the pinholes.

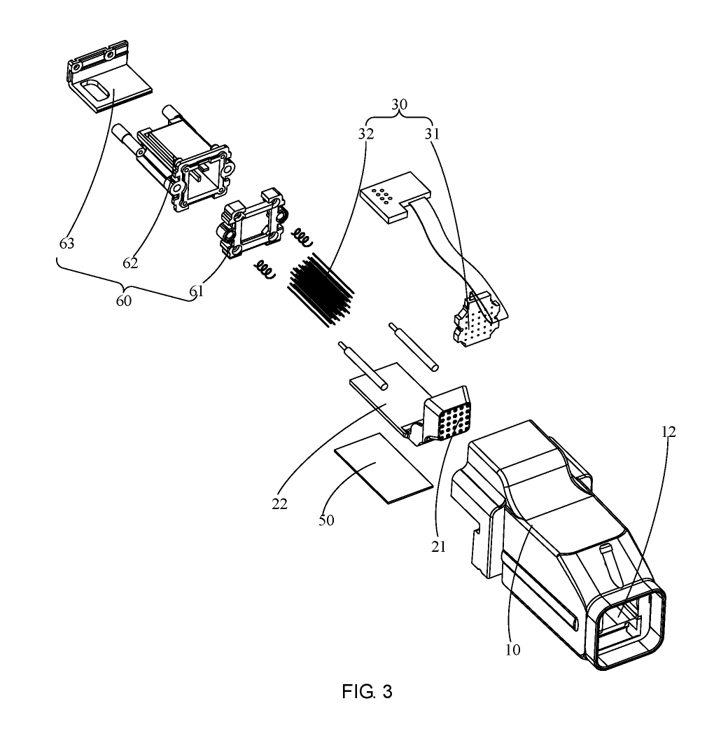

As shown in FIGS. 2-3, the contact end 21 is provided with the pinholes 212 for the microneedles 32 to thread. The microneedles 32 extend from the treatment port 12 through the pinholes 212 in the contact end 21, thus the treated area pierced by the microneedles 32 can receive better cold treatment, and the contact end 21 can support the microneedles 32 to avoid buckling to some extent. Thus the microneedles 32 can pierce into the treated area better. Of course, the microneedles 32 can extend from the treatment port 12 from the side of the contact end 21.

The contact end 21 has an outward-facing contact area 211, which is flushed with the front end of the treatment port 12. Thus, loose contact between the area that needs to be treated and the contact area 211 due to the contact area 211 being inside the treatment port 12 can be avoided.

As shown in FIG. 3, the heat-conducting end 22 is located at the edge of the contact end 21, and extends to the opposite direction from the treatment port 12. Because the microneedles 32 is inside the contact end 21, by means of locating the heat-conducting end 22 at the edge of the contact end 21, disturbance of the heat-conducting end 22 to the microneedles 32 can be avoided.

The microneedle base 30 is installed inside the receiving chamber 11 and its movement is adjustable, so that the microneedles can extend out of and retract from the treatment port 12. Installing the microneedle base 31 with adjustable movement inside the receiving chamber 11 enables the microneedles 32 to extend out of or retract from the pinholes 212. As such, the microneedles 32 can be controlled to pierce into the area that needs to be treated. Time, depth and frequency etc. of piercing can be selected based on the treatment schedule. The microneedles 32 can be used in combination with cold treatment on the area that needs to be treated. For example, cold treatment can be done before piercing, or vice versa. Such arrangement can keep the microneedles 32 from being easily damaged when they are always extending out of the pinhole 212.

As shown in FIG. 3, the microneedle base 31 is disposed to be able to slide toward and from the treatment port 12. The slidable arrangement of the microneedle base 31 is more beneficial for controlling the microneedles 32. Of course, other methods can also be adopted, such as using the way of screw rotation to move in a left-right direction. In some other embodiments, the microneedle treatment head is also provided with a supportive device 60, which includes a fixed base 61, a sliding housing 62 and a back stand 63. The support device 60 is for enabling the microneedle base 31 to slide more stably by fixing the microneedle base 31 between the fixed base 61 and the sliding housing 62.

As shown in FIGS. 1-2, a negative pressure chamber 40 is provided in the receiving chamber 11 around the interior side of the treatment port 12. The negative pressure chamber 40 is disposed with an open end toward the treatment port 12 for adsorbing the area that needs to be treated. The contact end 21 is disposed in the center of the treatment port 12. The negative pressure chamber 40 extends along the interior periphery of the treatment port 12. The negative pressure chamber 40 is connected with a negative pressure through-hole 41 for connection with a negative pressure output portion (not shown in the figures).

When using the microneedle treatment head 100, the negative pressure through-hole 41 is connected with the negative pressure output portion to produce negative pressure inside the negative pressure chamber 40. Meanwhile, the area that needs to be treated fits to the contact area 211 to block off the pinholes 212 of the contact area 211. Thus, an enclosed space can be formed in the negative pressure chamber 40, and the area that needs to be treated will be tightly adsorbed. Therefore, even the area that needs to be treated is soft, such as the skin in the neck, it is tightly adsorbed so that the microneedles 32 can easily pierce into the skin of the area that needs to be treated. Besides, the negative pressure chamber 40 can enable the area that needs to be treated to abut against the contact area 211 tightly, which is conducive to cold treatment of the area that needs to be treated. Moreover, since the contact area 211 flushes with the front end of the treatment port, loose suck from the negative pressure chamber 40 to the area that needs to be treated when the contact area 211 juts out from the treatment port 12 can be avoided, and the appearance of the microneedle treatment head 100 is more smooth and beautiful.

The contact end 21 is located at the center of the treatment port 12. The negative pressure chamber 40 extends along the interior periphery of the treatment port 12. Such arrangement can ensure more uniform adsorption from the negative pressure chamber 40 to the area that needs to be treated. Thus better effect of cold treatment and easier piercing of the microneedles 32 can be achieved.

As shown in FIG. 2, the negative pressure through-hole 41 runs through the housing 10 at the treatment port 12. Establishing the negative pressure through-hole 41 on the housing 10 at the treatment port 12 can directly connect the negative pressure chamber 40 to the negative pressure output portion from the outside of the housing 10. As such, the arrangement of a connecting pipe between the negative pressure through-hole 41 and the negative pressure output portion inside the receiving chamber 11 can be avoided, so that the space inside the receiving chamber 11 can be saved and the disturbance to the microneedle device 30 can be avoided.

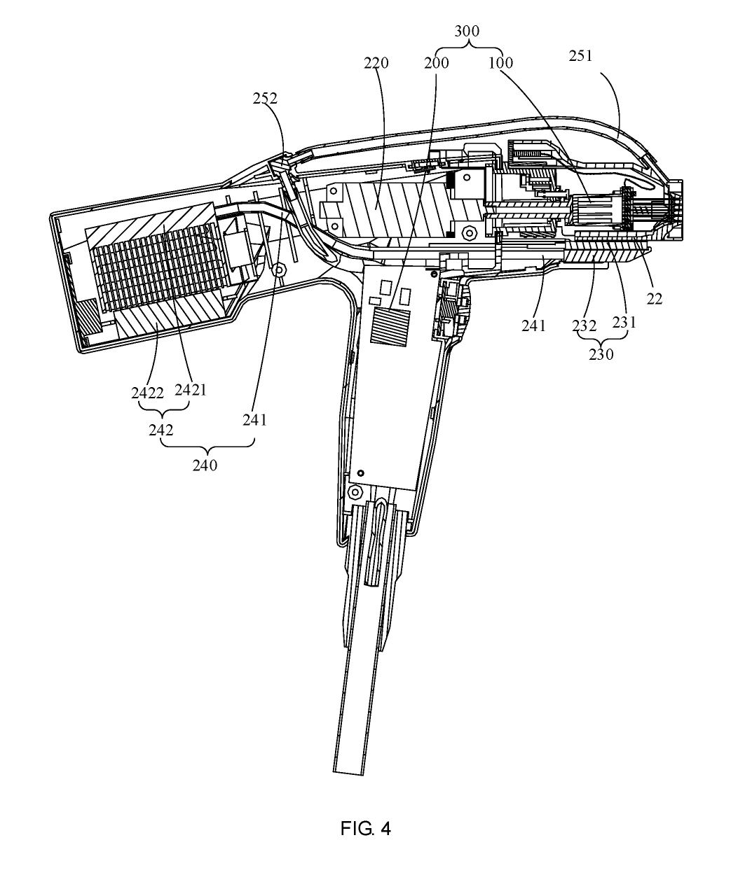

The present disclosure also provides a microneedle treatment apparatus 300, as shown in FIG. 4. The microneedle treatment apparatus 300 includes a treatment device body 200 and the microneedle treatment head 100. The concrete structure of the microneedle treatment head 100 refers to the above embodiments. Since, in some embodiments of the microneedle treatment apparatus 300, technical proposals in all the above embodiments are used, thus at least all the advantageous effect of the technical proposals in the above embodiments can be achieved, and detailed description will not be described here. The treatment device body 200 includes the cooling output portion which is connected with the heat-conducting end 22, so that the heat of the contact end 21 can be transferred to the cooling output portion. In some embodiments, the cooling output portion is disposed at the end of the treatment device body 200 to avoid an external connection of the microneedle treatment apparatus to other cooling output portions, thus the use of microneedle treatment apparatus is more convenient.

The cooling output portion includes a semiconductor cooling assembly 230 and a radiator 240. A cold end 231 of the semiconductor cooling assembly 230 is connected with the heat-conducting end 22. A hot end 232 of the semiconductor cooling assembly 230 is connected with the radiator 240. The cooling output portion achieves cooling by the semiconductor cooling assembly 230. The radiator 240 includes a radiator fan (not shown in the figures), a heat-conducting pipe 241 and a heat sink 242.

The hot end 232, which is connected with the cold end 231, conducts heat to a heat sink bottom 2421 through the heat-conducting pipe 241, and the radiator fan is used to help the heat dissipation of the heat sink 242. The heat-conducting pipe 241 can function as a heat transfer and red copper is used in some embodiments. The function of a heat transfer demands that the heat-conducting pipe 241 have good thermal conductive effect.

In some embodiments, a liquid-vapor heat-conducting pipe is used for the heat-conducting pipe 241. The liquid-vapor heat-conducting pipe includes a shell, a wick and an end cover. After achieving negative pressure through exhausting the air in the heat-conducting pipe 241, operating fluid is perfused into the heat-conducting pipe 241. After the wick capillary porous material, which was tightly attached to the inside of the heat-conducting pipe 241, is fully filed with the operating fluid, the heat-conducting pipe 241 is sealed. One end of the heat-conducting pipe 241 is the evaporation section (connected with the hot end 232 of the semiconductor cooling assembly 230), while the other end is the condensation section (connected with the heat sink 242). When the evaporation section of the heat-conducting pipe 241 is heated, the liquid in the capillary wick vaporizes and the steam flows to the condensation section under small differential pressure, giving off heat and congeals into liquid. Then the liquid flows back to the evaporation section along the porous material by capillary force. Repeating this circulation continuously, the heat is transferred to the condensation section from the evaporation section in the heat-conducting pipe 241.

A heat sink top 2422 is flaked metal which fully exchanges heat with air. The radiator fan sends off hot air quickly into the environment, which causes a bigger temperature difference between the heat sink 242 and the surrounding air to conduce a quicker heat exchange. In order to ensure better heat exchange and achieve lower temperature in the treatment area, more than one such as three to five heat-conducting pipes 241 may be needed.

A thermal conductive silicone 50 is disposed between the contact area in which the cold end 231 of the semiconductor cooling assembly 230 abuts against the heat-conducting end 22. Thus, the contact between the heat-conducting end 22 and the cold end 231 is more closed, so that better heat exchange effect can be achieved between them.

The treatment device body 200 is detachably connected with the microneedle treatment head 100. In some embodiments, the treatment device body 200 includes a drive portion for sliding the microneedle device to the left and right. As shown in FIG. 4, in these embodiments, the drive portion includes a pushrod portion 220, which further includes a pushrod. The pushrod can cross the installation port 13 in the left and right sides through an electric motor to move back and forth, so as to push the microneedle base 31 to slide to the left and right. Thus the microneedles 32 can extend from and retract to the pinholes 212. The treatment device body 200 is set to a handle shape for easy holding and use. The connection method between the microneedle treatment head 100 and the treatment device body 200 can be fastening, screw jointing, etc.

The treatment device body 200 is also equipped with the negative pressure output portion which is connected to the negative pressure chamber 40 through the negative pressure through-hole 41. In some embodiments, the negative pressure output portion includes a negative pressure pump, a filter 252 and a negative pressure connecting pipe 251. The air in the negative pressure chamber 40 is extracted by the negative pressure pump through the filter 252 via the negative pressure connecting pipe 251, so that negative pressure is produced in the negative pressure chamber 40.

The present disclosure also provides a variable frequency RF treatment system. It should be noted that the illustrations provided in the following examples only show the basic concept of some embodiments in the present disclosure in a schematic manner, so only the components related to the present disclosure are displayed in the diagram instead of being drawn according to the number, shape and size of components in actual implementation. During actual implementation, the form, number and proportion of each component can be changed randomly, and the layout of the components can be more complex.

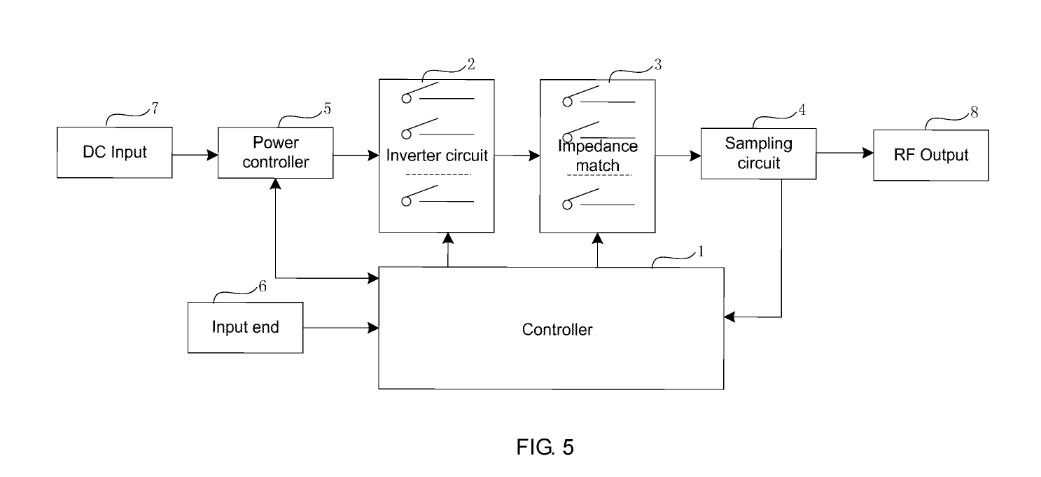

Referring to FIG. 5, which provides the structure diagram of the variable frequency RF treatment systemin some embodiments of the present disclosure. The variable frequency RF treatment system includes an inverter circuit 2 with various frequency outputs, an impedance matching circuit 3 and a controller 1. After a power signal is input in the inverter circuit 2, the inverter circuit and the impedance matching circuit are connected and they are both connected to the controller. The inverter circuit 2 switches the frequency according to the frequency conversion signal transmitted by the controller 1. According to the received actual impedance, the impedance matching circuit matches the optimal impedance and conducts the radiofrequency output.

The inverter circuit includes a plurality of switches, each correspondingly connecting to a fixed frequency. Power signal with different frequency can be output by closing the corresponding switch to change frequency. An optimal impedance can be matched by the impedance matching circuit according to the actual impedance of the skin tissue collected by the acquisition circuit (i.e., the impedance value is closest to or equal to the actual impedance value).

In some embodiments, the default resistance value of the impedance matching circuit is 50 ohms. The selection switch which is correspondingly connected to 50 ohms is closed to enable the output resistance value to be 50 ohms. The default frequency value of the inverter circuit is 1 MHZ. The switch which is correspondingly connected to 1 MHZ is closed to enable 1 MHZ frequency output. An input end 6 inputs a duty cycle pulse signal to the controller 1, and a DC input 7 supplies power to a power controller 5.

The controller 1 and the power controller 5 communicate with each other. The controller 1 sends a power control signal to adjust the power of the power controller 5 mainly by controlling the size of the DC current flowing through it. The controller 1 controls a RF output 8 to output radiofrequency waves for treatment according to the duty cycle of the pulse signal. The inverter circuit 2, which controls the output frequency of the power, switches the frequency of the power signal after receiving the frequency conversion signal from the controller 1. The impedance matching circuit 3 controls the impedance output by receiving the impedance matching signal from the controller 1.

The various embodiments of the variable frequency RF treatment system provided in the present disclosure can not only carry out radiofrequency treatment on different skins and detect the actual impedance value of the skin tissue, but also switch to an optimal output frequency according to different skin conditions being treated, thus improving the treatment effect and the treatment precision.

In some embodiments of the present disclosure, the circuit modulation mode is adopted. In the sampling process, after a short pulse is input, the sampling circuit 4 can calculate the actual impedance by detecting the current and voltage flowing through the tissue. For example, the matching output power of the impedance circuit is P1=I²R1, which is different from the output power of the actual impedance P2=I²R2 and the difference ratio is R2/R1. By adjusting the magnitude of the input DC current, the same power P can be output. Generally, the aforementioned calculation mode of impedance matching may cause inefficient radiofrequency output. Especially when it operates at full capacity, or when the impedance difference is too large, the output power required by the target cannot be achieved. Meanwhile, a series of equipment risks can be caused by long-term full-load or overload operation.

In some embodiments of the present disclosure, the modulation mode of impedance circuit (impedance matching circuit) is employed, where the sampling circuit is used for detecting the actual impedance of the skin tissue. When the actual impedance of the skin tissue is acquired, the impedance matching circuit is adjusted to output an impedance value equal to or very close to the actual impedance. The impedance matching circuit is provided with a plurality of selection switches, each correspondingly connected to an impedance value.

Wherein, the number of the selection switches is preferably 1-50. The larger the number is, the higher the precision of the matching impedance output is, the closer to the actual resistance value of impedance. Due to the fact that the conventional impedance of the human body tissue is about 10-500 ohms, and the number of impedance matching circuits is limited, it is impossible to output the impedance value which is exactly the same as the actual impedance at all time. Generally, the corresponding impedance divisional value on one line may range from 5 ohms, 10 ohms, 20 ohms to 50 ohms, and there may be 1-50 groups of lines. The resolution of the impedance matching line is high in the impedance concentration area or when the impedance is small. The resolution of the impedance matching line is low when in the area of large impedance.

For example, in the small impedance area, the divisional resistance value of the matching circuit is 1 ohm, while in the large impedance area, the divisional resistance value of the matching circuit area is 50 ohms. Thereinto, the impedance matching circuit, according to the actual impedance of the sampling circuit received by the controller, can calculate and determine which selection switch or switches need(s) to be closed, so that the closest or most appropriate impedance can be output.

In some embodiments, impedance matching is performed by combining the current modulation with switching the impedance matching circuit. The impedance circuit matching is employed to output a resistance value closest to the actual impedance. The ratio of the resistance value between the actual impedance and the output impedance is calculated. The error indicated by the ratio of the resistance values is adjusted by adopting the mode of current modulation and in the result, the same power value (P1=P2) is output. The accuracy of impedance matching can be further improved when the jointly coordinated regulation is made according to the aforementioned method, so that the impedance matching circuit can match with an optimal impedance value, therefore improving the efficacy and the precision of treatment.

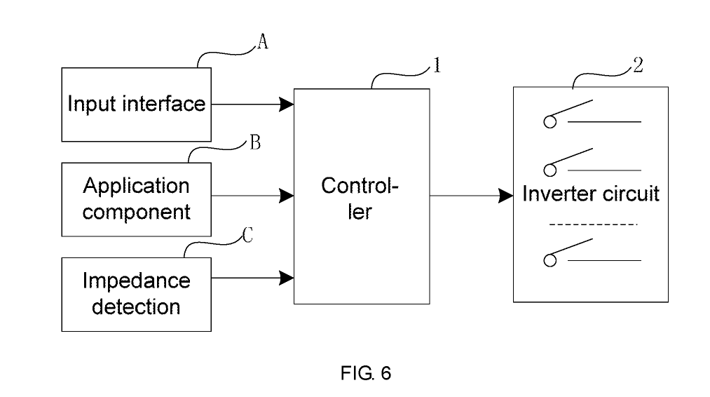

FIG. 6 shows a structure diagram of a frequency output control provided in some embodiments of the present disclosure, wherein three modes, including an input interface A, an application portion B and an impedance detection C, are used to control frequency. First, different application circuits need to be used for treating different tissue areas. In order to quickly obtain an optimal frequency for treating different tissue, generally the frequency output specified by the application portion can be used as a default, so that the treatment effect of clinical indications is improved. Second, the clinical indications of the user should be determined before treatment and according to the different clinical indications, select a superior frequency output.

Particularly for medical staff or users who know about their illness, the input interface can be used for selecting the needed output frequency to perform accurate treatment on the clinical indications. Third, when the application portion is used for tissue treatment, difference exists due to different conditions of the tissue. For example, when the surgery is performed to treat coagulation necrosis of fat, because of different dosages of the injected swelling anesthesia, the impedance is remarkably different, and the thermal dispersion capability is also different. In order to obtain matching impedance, the sampling circuit needs to detect the specific conditions of the user's skin tissue in real time so that the impedance change of the tissue can be automatically detected when it changes and the output frequency can be adjusted to obtain the required output frequency of the target. At the same time, automatic frequency switching is often achieved with the cooperation of the impedance detection mode.

To adapt to the situation where the impedance of the tissue keeps changing, calculation of impedance matching will be carried out after secondary sampling or repeated sampling even though the frequency has been changed. The inverter circuit includes a plurality of switches, each correspondingly connected to a fixed frequency. When a certain switch is closed, the corresponding frequency is output. In some embodiments, the number of the switches is 1-10, preferably 5. Switch can be added or deleted according to the user's requirement. In some embodiments, the common frequencies, which the switches are correspondingly connected to, are 300 KHZ, 460 KHZ, 800 KHZ, 1 MHZ, 2 MHZ and 2.45 MHZ, 3 MHZ, 4 MHZ, 6 MHZ, 6.78 MHZ, 10 MHZ and et al.

In some embodiments, among the three modes of controlling output frequency, the third impedance matching mode is adopted. Since different optimal frequencies need to be used for treating different clinical indications and most users do not know how to operate the RF treatment system or have no idea what kind of frequency or power should be used to treat different skin tissues to achieve the best treatment efficacy, usually treatment with appropriate output frequency according to different conditions of skin tissues is the best choice, which can also obtain the optimal treatment efficacy. For example, in the treatment for dermal tension with microneedle array, the optimal frequency corresponding to the impedance is 1-2 MHZ; in the treatment for the coagulation necrosis of fat with microneedle array, in order to ensure a large area of thermal dispersion, the optimal frequency corresponding to the impedance is 460 KHZ; when the needle electrode is applied to a deep layer fat, the optimal frequency corresponding to its impedance is 1 MHZ, while the optimal frequency corresponding to its impedance is 2-4 MHZ when it acts on an upper layer fat or even the dermis and 6 MHZ when it is used for nerve stripping. All the aforementioned examples directly or indirectly demonstrate that effective treatment and optimal efficacy can only be achieved when radiofrequency waves with different frequencies are used for treating different skin tissues.

In some embodiments of the present disclosure, when the RF treatment system starts its treatment, the default resistance value of the impedance matching circuit is 50 ohms, and the default frequency of the inverter circuit is 1 MHZ. The sampling circuit collects the actual impedance of the user's skin tissue and inputs the data to the controller which selects the corresponding frequency according to the received actual impedance.

At this time, the power signal of the default power is switched to the corresponding fixed frequency according to the frequency conversion signal sent by the controller so that a power signal with a fixed frequency is output. The power signal with the fixed frequency is loaded to an impedance matching the actual impedance and a precise radiofrequency treatment wave is output, which is more beneficial to the effective treatment for clinical indications. Thereinto, the mode of matching the actual impedance, as described above, will not be repeated here.

Some embodiments of the present disclosure can not only carry out RF treatment on different skins and detect the impedance value of the skin tissue, but also switch to the optimal output frequency according to the different skin conditions, thus improving the treatment effect and the treatment precision. Therefore, the present disclosure effectively overcomes various defects of existing technologies and enjoys a high industrial utilization value.

In some embodiments, a treatment system is provided including the variable frequency RF treatment circuits described above, and the microneedle treatment apparatus described above. Variable frequency RF electromagnetic field can be delivered to the patent through the microneedles to achieve treatment effects. Cooling can be performed at the same time through the treatment head to avoid thermal damages to the skin or tissues.

Although specific embodiments have been described above in detail, the description is merely for purposes of illustration. It should be appreciated, therefore, that many aspects described above are not intended as required or essential elements unless explicitly stated otherwise. Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. A microneedle treatment head, comprising:
a housing having a receiving chamber and a treatment port communicating with the receiving chamber;
a microneedle device disposed in the receiving chamber and comprising a microneedle base and at least one microneedle, wherein the microneedles are fixed to the microneedle base and extend toward the treatment port; and
a cold end conducting portion fixed in the receiving chamber and including a contact end exposed to the treatment port and a heat-conducting end which connects to the contact end with thermal conductive connection, wherein the heat-conducting end is connected to a cold output end of a cooling output device and the connection is thermal conductive.

2. The microneedle treatment head of claim 1, wherein the contact end comprises pinholes pierced by the microneedles.

3. The microneedle treatment head of claim 1, wherein the contact end has an outward-facing contact area which is aligned with the treatment port.

4. The microneedle treatment head of claim 1, wherein the microneedle base is adjustably installed inside the receiving chamber, so that the microneedle can extend from and retract to the treatment port.

5. The microneedle treatment head of claim 1, wherein the treatment port is provided with a negative pressure chamber which is disposed toward one side of the treatment port with an open end; the contact end is disposed in the center of the treatment port and the negative pressure chamber extends along the periphery of the treatment port; and the negative pressure chamber is provided with a negative pressure through-hole for connection with a negative pressure output portion.

6. A microneedle treatment apparatus, comprising a microneedle treatment head of claim 1 and a treatment device body;
wherein the microneedle treatment head is fastened to the treatment device body which comprises the cooling output portion;
wherein the heat-conduction end has cold conduction connection with the cooling output portion.

7. The microneedle treatment apparatus of claim 6, wherein the cooling output portion comprises a semiconductor cooling assembly and a radiator;
wherein, a cold end of the semiconductor cooling assembly is connected to the heat-conduction end and a hot end of the semiconductor cooling assembly has a cold conduction connection with the radiator.

8. The microneedle treatment apparatus of claim 7, wherein the radiator comprises a heat-conducting pipe and a heat sink;
wherein, the heat-conducting pipe is connected to the hot end of the semiconductor cooling assembly for conducting the heat of the hot end of the semiconductor cooling assembly to the heat sink;
wherein the heat-conducting pipe is a liquid-vapor heat pipe.

9. The microneedle treatment apparatus of claim 6, wherein the treatment device body is detachably connected with the microneedle treatment head; wherein, the treatment device body comprises a drive portion which is connected to the microneedle base.

10. The microneedle treatment apparatus of claim 6, wherein the treatment device body comprises a negative pressure output portion, which is connected to the negative pressure chamber through a negative pressure through-hole.

11. A variable frequency RF treatment system, comprising:
an inverter circuit with various frequency outputs;
an impedance matching circuit; and
a controller;
wherein after a power signal is input in the inverter circuit, the inverter circuit and the impedance matching circuit are connected and they are connected to the controller;
the inverter circuit switches the frequency according to the frequency conversion signal transmitted by the controller; and
when the impedance matching circuit matches an optimal impedance according to a received actual impedance, radiofrequency is output.

12. The variable frequency RF treatment system of claim 11, wherein the inverter circuit comprises a plurality of switches, each correspondingly connecting to a fixed frequency.

13. The variable frequency RF treatment system of claim 11, wherein the impedance matching circuit performs impedance matching by switching to different impedance matching circuits; wherein the impedance matching circuit comprises multiplexer switches, each correspondingly connecting to an impedance.

14. The variable frequency RF treatment system of claim 11, wherein the impedance matching circuit performs impedance matching by current modulation.

15. The variable frequency RF treatment system of claim 11, wherein the impedance matching circuit performs impedance matching by combining impedance matching circuits and current modulation.

16. The variable frequency RF treatment system of claim 11, comprising an acquisition circuit and an actual impedance from user's skin tissues acquired by the acquisition circuit is sent to the controller.

17. The variable frequency RF treatment system of claim 11, wherein the power signal is generated by a power controller which connects the controller to receive its setting.

18. The variable frequency RF treatment system of claim 11, wherein frequencies are set respectively on the basis of different designations of application portions.

19. The variable frequency RF treatment system of claim 11, wherein the frequency is set on the basis of an input of a user interface.

20. The variable frequency RF treatment system of claim 11, wherein the frequency is set on the basis of the detection mode of impedance matching.
